(19) United States
(12) Patent Application Publication
(10) Pub. No.: US 2015/0307418 A1
(43) Pub. Date: Oct. 29, 2015

(54) METHODS AND APPARATUSES FOR PRODUCING XYLENE FROM LIGNIN

(71) Applicant: UOP LLC, Des Plaines, IL (US)

(72) Inventors: Stanley Joseph Frey, Palatine, IL (US); Joao Jorge da Silva Ferreira Alves, Bramley (GB)

(73) Assignee: UOP LLC, Des Plaines, IL (US)

(21) Appl. No.: 14/260,176

(22) Filed: Apr. 23, 2014

(51) Int. Cl. C07C 7/10; C07C 4/22; C07C 7/12; C07C 5/27; C07C 5/32; C07C 6/12

(52) U.S. Cl. CPC C07C 7/10; C07C 5/321; C07C 6/12; C07C 7/12; C07C 5/2702; C07C 4/22

(57) ABSTRACT

Methods and apparatuses are provided for producing a xylene product from a lignin supply. A method includes depolymerizing the lignin supply to produce a lignin aromatic stream, and isomerizing the lignin aromatic stream to produce an isomerized lignin stream. The desired xylene isomer is extracted from the isomerized lignin stream.

### TECHNICAL FIELD

The present disclosure generally relates to systems and methods for producing xylene from lignin, and more particularly relates to systems and methods for recovering a desired isomer of xylene from lignin.

### BACKGROUND

Xylene isomers are important intermediates in chemical syntheses, and specific xylene isomers are desired for different processes. Paraxylene is a feedstock for terephthalic acid production, and terephthalic acid is used in the manufacture of synthetic fibers and resins. Metaxylene is used in the manufacture of certain plasticizers, azo dyes, and wood preservatives. Orthoxylene is a feedstock for phthalic anhydride production, and phthalic anhydride is used in the manufacture of certain plasticizers, dyes, and pharmaceutical products. Desired xylene isomers are typically extracted from petroleum feedstocks, but the increasing demand for specific xylene isomers creates supply pressures on the petroleum feedstocks.

An aromatics complex produces one or more xylene isomers, alongside with benzene and often toluene. Such an aromatics complex may include a transalkylation process unit. The transalkylation process unit can increase the yield of xylenes in the aromatics complex because it converts toluene and aromatics with 9 to 11 carbons into 8 carbon xylene compounds and benzene. However, alkyl groups with 2 or more carbon atoms, such as ethyl, propyl and butyl groups, tend to be severed from the aromatic ring during transalkylation such that benzene and light paraffins are produced. This reduces the overall combined yield of xylenes and benzene due to the long chain alkyl groups being dealkylated and being deposited in the low value fuel gas or liquefied petroleum gas (LPG) pool. Feeds, rich in alkyl groups with 2 or more carbon atoms, may produce very little xylene at all.

Lignin is a polymer produced in large quantities by woody plants. Lignin is a solid, but it can be solubilized and depolymerized to produce liquid products. There are typically several different compounds in liquid products produced from lignin, including high concentrations of higher alkyl benzene compounds, where the higher alkyl groups include ethyl, propyl, and butyl benzene compounds, but small quantities of benzene with attached methyl groups. Transalkylation of the higher alkyl benzene compounds from depolymerized lignin tends to produce benzene, LPG, and fuel gas with relatively low production of xylene compounds.

Accordingly, it is desirable to develop apparatuses and methods for converting higher alkyl benzenes into xylenes. In addition, it is desirable to develop apparatuses and methods for producing xylenes from reproducible natural feedstocks, such as woody plants. Furthermore, other desirable features and characteristics of the present embodiment will become apparent from the subsequent detailed description and the appended claims, taken in conjunction with the accompanying drawings and this background.

### BRIEF SUMMARY

A method is provided for producing a desired xylene isomer from lignin. The method includes depolymerizing a lignin supply to produce a lignin aromatic stream, and isomerizing the lignin aromatic stream to produce an isomerized lignin stream. The desired xylene isomer is extracted from the isomerized lignin stream.

In another embodiment, a method is provided for producing xylene from lignin. The method includes depolymerizing lignin to produce a lignin aromatic stream, and hydrotreating the lignin aromatic stream to remove oxygen and create a hydrotreated lignin aromatic stream. The hydrotreated lignin aromatic stream is contacted with an isomerization catalyst to produce an isomerized lignin stream, and xylene is extracted from the isomerized lignin stream.

An apparatus is also provided for producing a desired xylene isomer from lignin. The apparatus includes a lignin depolymerization unit configured to produce a lignin aromatic stream, and an isomerization unit configured to accept the lignin aromatic stream. The isomerization unit is further configured to produce an isomerized lignin stream, and a dehydrogenation unit is configured to accept the lignin aromatic stream, where the dehydrogenation unit is also configured to produce a methyl enhanced aromatics stream. An aromatics complex is configured to accept the methyl enhanced aromatics stream. The aromatics complex includes a xylene recovery unit, a transalkylation unit, and a xylene isomerization unit, where the xylene recovery unit is configured to extract the desired xylene isomer.

### BRIEF DESCRIPTION OF THE DRAWINGS

The present embodiment will hereinafter be described in conjunction with the following drawing figures, wherein like numerals denote like elements, and wherein:

FIG. 1 is a schematic diagram of an exemplary embodiment of a xylene production apparatus and method for producing xylene from lignin;

FIG. 2 illustrates a lignin polymer and a product from depolymerization of the lignin polymer;

FIG. 3 is another embodiment of a xylene production apparatus and method for producing xylene from lignin; and

FIG. 4 is yet another embodiment of a xylene production apparatus and method for producing xylene from a lignin aromatic stream.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The various embodiments described herein relate to systems and methods for producing a desired xylene isomer from a lignin supply. The lignin supply is depolymerized to produce a lignin aromatic stream, where the lignin aromatic stream is rich in propyl benzene and other multi-carbon alkyl aromatics, and has a low concentration of aromatic compounds with attached methyl groups. The term “rich in propyl benzene” means greater than about 10 mole percent or more of the aromatic compounds with 9 carbon atoms are propyl benzene. In some embodiments, propyl benzene may be present in the lignin aromatic stream at greater than about 40 mole percent or more of the aromatic compounds with 9 carbon atoms. The low concentration of aromatic compounds with attached methyl groups means there are relatively few aromatic compounds with a specific number of carbon atoms that include a methyl group pendent from a benzene ring, where the specific number of carbon atoms is 8 or more. Multi-carbon alkyl aromatics are aromatic compounds with attached alkyl groups having more than one carbon, such as ethyl benzene, propyl benzene, and butyl benzene. The lignin aromatic stream is isomerized using a catalyst that isomerizes multi-carbon alkyl aromatics to produce an equilibrium mixture that increases the number of aromatic compounds with methyl groups. The isomerization process increases the concentration of aromatic compounds with attached methyl groups in the resulting isomerized lignin stream, but some aromatic compounds are converted to naphthenes as well. The isomerized lignin stream may be dehydrogenated to convert the naphthenes back into aromatic compounds in a methyl enhanced aromatic stream. The methyl enhanced aromatic stream is then introduced into an aromatics complex where the aromatic compounds having 9 or more carbon atoms and also having methyl groups are transalkylated with toluene to produce xylenes, and may produce other compounds as well. A desired xylene isomer is recovered, and benzene and other compounds produced from the lignin supply may also be collected. This process produces valuable xylenes and other products from a lignin supply.

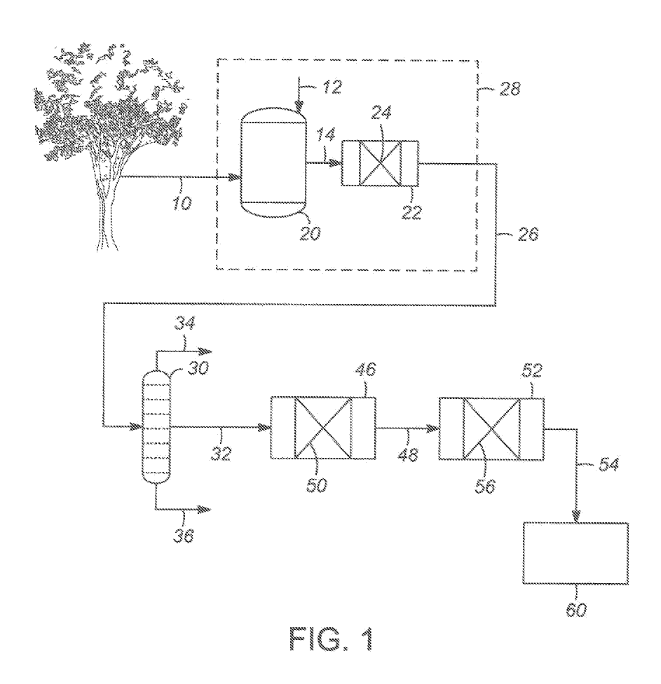

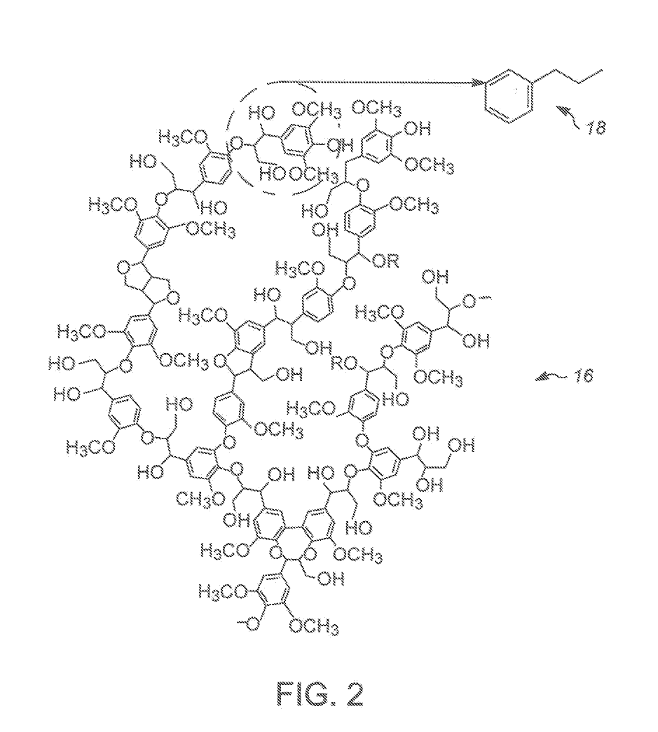

An exemplary method and apparatus will now be described with reference to FIG. 1. In this embodiment, a lignin supply 10 is combined with a lignin solvent 12 to create a lignin slurry 14. Lignin for the lignin supply 10 is most commonly available from wood, which is a naturally occurring and renewable resource, but it is also present in the secondary cell walls of many plants and some algae, such as straw, corn stover, and bagasse. Lignin may be obtained from paper mills, saw mills, farm harvests, or a wide variety of other sources. Referring momentarily to FIG. 2, with continuing reference to FIG. 1 and without intending to be limiting, an example of the polymeric form of lignin 16 is illustrated, and FIG. 2 further illustrates how removal of oxygen atoms from lignin 16 during depolymerization can produce propyl benzene 18. In general, lignin is a polymer including para-hydroxyphenyl units, syringyl units, and guaiacyl units.

The lignin solvent 12 may be water in a liquid form, and the lignin solvent 12 may also include a hydrogen donor such as acids, glycols, other alcohols, or other hydrogen donors, including but not limited to ethylene glycol, propylene glycol, other glycols, sulfuric acid, hydrochloric acid, nitric acid, acetic acid, citric acid, and formic acid. Many other materials can be used as the lignin solvent 12 in other embodiments, such as aromatic hydrocarbons, including but not limited to benzene, toluene, xylenes and/or fused ring aromatic compounds such as tetralin and its alkylated derivatives. Additional compounds may also be optionally added to the lignin slurry 14, such as surfactants or emulsifying agents. In some embodiments, the lignin 16 is dissolved in the lignin solvent 12 to produce a solution, but in many embodiments some of the lignin 16 is not completely dissolved to produce the lignin slurry 14.

In some embodiments, the lignin supply 10 is pretreated (not illustrated), such as with steam explosion, hammer milling, grinding, chipping or other size reduction, soaking in the lignin solvent 12 or in other solvents, saturation with steam, pressurizing at pressures from about 1 atmosphere to about 150 atmospheres, enzymatic hydrolysis, alkaline wet oxidation, other methods, or combinations thereof. Preliminary soaking may be at elevated temperatures in some embodiments, such as from about 120 degrees centigrade (° C.) to about 250° C., and may last from about 1 minute to about 24 hours or longer. The lignin 16 is often present with other compounds in plants, such as cellulose and hemicellulose, and the lignin 16 may or may not be separated from other components in the pretreatment step or steps.

The lignin slurry 14 may be formed in a lignin slurry vessel 20. In some embodiments, the particle size in the lignin slurry vessel 20 may be reduced to about 200 microns or less, such as with steam explosion or with high shear forces. The particle size may be reduced during the pretreatment step, if conducted, or in the lignin slurry vessel 20. In other embodiments, larger particle sizes are allowed. The particle size of the lignin slurry 14 may be reduced before or after adding the lignin solvent 12 to the lignin supply 10 in the lignin slurry vessel 20, and a partial vacuum may be applied to the lignin supply 10 to aid in dispersing solids in the lignin solvent 12 for particle size reduction.

The lignin slurry 14 is transferred to a lignin reactor 22 containing a lignin catalyst 24, where the lignin slurry 14 is depolymerized to form a lignin aromatic stream 26. The lignin slurry vessel 20 and the lignin reactor 22 form a lignin depolymerization unit 28, but the lignin depolymerization unit 28 may have other vessels in alternate embodiments. Hydrogen is added to the lignin slurry 14 in the presence of the lignin catalyst 24, and the hydrogen reacts with oxygen in the polymeric lignin 16 to break the carbon/oxygen bonds and thereby depolymerize the lignin 16. The hydrogen may be introduced to the lignin reactor 22 by a variety of methods or techniques, including hydrogen donor compounds in the lignin slurry 14 or as gaseous hydrogen. The lignin reactor 22 may be more than one reactor in some embodiments, and there may be more than one type of lignin catalyst 24 used in the same or different reactors. Different lignin reactors 22 may be operated in series or in parallel, and there may be different temperatures, pressures, and residence times in different reactors as well.

The lignin slurry 14 is contacted with hydrogen in the presence of the lignin catalyst 24 under conditions which promote the depolymerization of lignin to aromatic oxygenate substituents (e.g., phenol, and its alkoxylated derivatives) and also the deoxygenation (or hydrodeoxygenation) of these substituents or intermediates to aromatic hydrocarbons. The lignin catalyst 24 may be present in the form of solid particles with a catalytically active metal disposed on a support, or in the form of a compound containing the catalytically active metal. The lignin catalyst 24 may include at least one Group VIII metal for a hydrogenation function, such as iron, cobalt, nickel, or combinations thereof, and/or one or more Group VI metals, such as molybdenum and tungsten. Noble metals, such as ruthenium, palladium, and platinum, may also be used for a hydrogenation function. A representative lignin catalyst 24 includes a metal selected from ruthenium, rhodium, platinum, palladium, iron, cobalt, nickel, molybdenum, tungsten, and mixtures thereof, and may include a support in some embodiments. The lignin catalyst 24 may also include an acid function, and the acid function may be imparted by the support or otherwise incorporated into the lignin catalyst 24. Representative acidic support materials that can serve as the acid function include clays (e.g., Minugel®, kaolin, kaolinites, halloysite, etc.), zeolites, non-zeolitic molecular sieves, mixed metal oxides, sulfated zirconia, and other materials that contain acid sites and that can be used in varying amounts to regulate the overall acidity of the catalyst. In alternate embodiments, the lignin catalyst 24 may be an acid, sodium hydroxide or other bases, or combinations of catalytically active metals with an acid or base. The lignin catalyst 24 may be in a fixed bed or moving bed, and homogeneous systems operating with catalysts that are soluble in the reactants and products may also be used.

Catalytic depolymerization/deoxygenation conditions will vary depending on the quality of the lignin conversion effluent desired, with higher severity operations resulting in greater conversion of organic oxygenate intermediates and other oxygenated species (e.g., carboxylic acids) by hydrogenation. Typical lignin depolymerization reaction conditions include a temperature of from about 40° C. to about 700° C., or from about 200° C. to about 600° C., or from about 190° C. to about 370° C. in various embodiments. If hydrogen gas is used to supply the hydrogen, the hydrogen partial pressure may be from about 6 atmospheres to about 300 atmospheres, or from about 80 atmospheres to about 250 atmospheres, or from about 50 atmospheres 150 atmospheres in various embodiments. If a hydrogen donor is used to supply the hydrogen, the hydrogen donor may be introduced into the lignin reactor 22 in the lignin slurry 14, or the hydrogen donor may be introduced separately, or the hydrogen donor may be introduced with the lignin slurry 14 and also added separately. In some embodiments, the reaction temperature is higher than the boiling point of the lignin solvent 12 at atmospheric pressure, but the reaction temperature is less than the critical temperature of the lignin slurry 14. The pressure in the reactor may be increased until the reaction proceeds without the formation of char, or carbonaceous residue, in the lignin reactor 22.

In addition to pressure and temperature, the residence time of the lignin slurry 14 and hydrogen in the lignin reactor 22 can also be adjusted to increase or decrease the reaction severity and consequently the quality of the resulting lignin aromatic stream 26. With all other variables unchanged, lower residence times are associated with lower reaction severity. For example, A Weight Hourly Space Velocity (WHSV, expressed in units of hr⁻¹) from about 0.1 hr⁻¹ to about 20 hr⁻¹ may be used, but in alternate embodiments a WHSV of from about 0.5 hr⁻¹ to about 10 hr⁻¹ may be used. The quantity of hydrogen used may be based on the stoichiometric amount needed to completely convert the oxygen present in the lignin 16 to water (H₂O). In representative embodiments, the reaction may be carried out in the presence of hydrogen in an amount ranging from about 90% to about 600% of the stoichiometric amount.

The polymeric form of lignin 16 does not have a specific form, but does include para-hydroxyphenyl units, syringyl units, and guaiacyl units, as mentioned above. As such, the depolymerized and deoxygenated compounds in the lignin aromatic stream 26 may include a relatively high concentration of N-propyl benzene compounds, and also tends to include other higher alkyl benzene compounds such as ethyl benzene and butyl benzene, as mentioned above. Propyl benzene can be referred to as an A9 compound, because it is an aromatic compound with 9 carbon atoms. In this description, “A9” represents aromatic compounds with 9 carbon atoms, where the letter “A” represents “aromatic” and the following number represents the number of carbon atoms in the compound. In a similar manner, A10 refers to aromatic compounds with 10 carbon atoms; “A7-” refers to aromatic compounds with 7 or fewer carbon compounds, and so on. In an exemplary embodiment, the lignin aromatic stream 26 includes several different compounds, including A8, A9, and A10 compounds, where about 50 mole percent or more of the A8 compounds are ethyl benzene, about 50 mole percent or more of the A9 compounds are n-propyl benzene, and about 50 mole percent or more of the A10 compounds are N-butyl benzene.

The lignin aromatic stream 26 may optionally be fractionated in a lignin fractionator 30 to produce a lignin center cut stream 32, a lignin light ends stream 34, and a lignin heavy ends stream 36. In an exemplary embodiment, the lignin fractionator 30 is operated such that the lignin light ends stream 34 includes toluene and lighter compounds (lighter than toluene), where reference to a “lighter” compound means a compound with a lower boiling point at atmospheric pressure than a reference compound (toluene in this case), and where a “heavier” compound means a compound with a higher boiling point at atmospheric pressure than a reference compound. The lignin fractionator 30 may also be operated such that the lignin heavy ends stream 36 includes naphthalene and heavier compounds (heavier than naphthalene), so the lignin center cut stream 32 includes compounds with boiling points between those of the lignin light ends stream 34 and the lignin heavy ends stream 36. In an exemplary embodiment, the lignin fractionator 30 is operated with the lignin light ends stream 34 exiting at a temperature of from about 100° C. to about 150° C. at a pressure of from about 0.8 atmospheres to about 3 atmospheres, and with the lignin heavy ends stream 36 exiting at from about 200° C. to about 300° C. and a pressure of from about 1 atmosphere to about 4 atmospheres, but other operating conditions are also possible. In alternate embodiments, the lignin aromatic stream 26 is not fractionated. The lignin aromatic stream 26 may be transferred to the lignin fractionator in a variety of ways, such as direct piping so the lignin depolymerization unit 28 is fluidly coupled to the lignin fractionator 30, or by other transport such as rail car, barge, truck, etc. The lignin depolymerization unit 28 is configured to produce the lignin aromatic stream 26, and lignin fractionator 30 is configured to accept the lignin aromatic stream 26.

In an exemplary embodiment, an isomerization unit 46 is fluidly coupled to the lignin depolymerization unit 28. In alternate embodiments, the lignin aromatic stream 26 or the portion of the lignin aromatic stream 26 present in the lignin center cut stream 32 is transported to the isomerization unit 46, such as by rail car, barge, truck, etc., so the isomerization unit 46 is configured to accept the lignin aromatic stream 26 or the portion of the lignin aromatic stream 26 present in the lignin center cut stream 32 in an appropriate manner. The lignin aromatic stream 26, or the portion of the lignin aromatic stream 26 present in the lignin center cut stream 32, is combined with hydrogen and contacted with an isomerization catalyst 50 in the isomerization unit 46 to produce an isomerized lignin stream 48. The term “isomerization” is used herein to describe the conversion of an aromatic hydrocarbon into at least one different aromatic hydrocarbon having the same number of carbon atoms. Isomerization desirably converts those aromatics having at least one ethyl, propyl, or butyl substitute, such as methyl-ethyl benzene and propyl benzene into an aromatic compound with more methyl substitutions. The availability of additional methyl groups increases the xylene yield from the lignin supply 10. In addition, the methyl groups on A9 compounds are highly stable at transalkylation reaction conditions, described below, and are essentially conserved in the transalkylation reaction. The higher alkyl benzene compounds are isomerized to form methylated benzene compounds at equilibrium concentrations, or at closer to equilibrium concentrations than in the lignin aromatic stream 26 or the lignin center cut stream 32. In this description, “methylated benzene compounds” or “methylated aromatic compounds” means compounds that include a benzene ring with one or more attached methyl groups.

The isomerization catalyst 50 can be in a fixed-bed system, a moving-bed system, a fluidized-bed system, or in a batch-type operation in the isomerization unit 46. The isomerization unit 46 may be one or more separate reactors configured to ensure that the desired isomerization temperature is maintained at the entrance to each reactor. The hydrocarbons from the lignin aromatic stream 26 may contact the isomerization catalyst 50 in a liquid phase, a mixed liquid vapor phase, or in a vapor phase in various embodiments.

The hydrocarbons from the lignin aromatic stream 26 contact the isomerization catalyst 50 at isomerization conditions, such as a temperature of from about 250° C. to about 450° C. and a pressure of from about 3 atmospheres to about 15 atmospheres. Sufficient isomerization catalyst 50 is contained in the isomerization unit 46 to provide a liquid hourly space velocity with respect to the hydrocarbon feed mixture of from about 0.1 to about 30 hr⁻¹, and preferably 0.5 to 10 h⁻¹. Hydrocarbons from the lignin aromatic stream 26 are reacted in admixture with hydrogen at a hydrogen/hydrocarbon mole ratio of about 0.5:1 to about 25:1 or more. Isomerization catalysts 50 are well known in the art, and many different types of isomerization catalysts 50 can be used. In one embodiment, the isomerization catalyst 50 includes an acid function to promote naphthenic ring transformation from cyclohexanes to cyclopentanes and back again, and a metal function to promote ethyl, propyl, and butyl group isomerization to substituted methyl groups. As such, the isomerization catalyst 50 may include a low Si/Al₂ MTW type zeolite, also characterized as “low silica ZSM-12.” The MTW-type zeolite may be composited with a binder for convenient formation of catalyst particles, where the binder may be a refractory inorganic oxide such as alumina. The isomerization catalyst 50 also includes a hydrogenation catalyst component, such as one or more of platinum, palladium, rhodium, ruthenium, osmium, and iridium (the platinum group metals), which may be present at about 0.01 to about 2 weight percent of the total isomerization catalyst 50. Other metals may also be present in various embodiments, including but not limited to tin, germanium, or lead. The metal component(s) may be in a variety of chemical forms, such as an oxide, sulfide, halide, oxysulfide, as an elemental metal, or in combination with one or more other ingredients of the catalyst composite.

Without being bound to any particular theory, it is believed that hydrocarbon compounds are partially saturated into naphthenic compounds in the isomerization unit 46, and carbon atoms on the higher alkyl groups move more freely in naphthenic compounds. The depolymerized lignin 16 in the lignin aromatic stream 26 has a high concentration of higher alkyl benzene compounds, as mentioned above, so the higher alkyl benzene compounds are present at more than the equilibrium concentration. As such, the isomerized lignin stream 48 has a higher concentration of methylated benzene compounds and a lower concentration of higher alkyl benzene compounds than the lignin aromatic stream 26.

The isomerized lignin stream 48 produced in the isomerization unit 46 is dehydrogenated by contacting a dehydrogenation catalyst 56 in a dehydrogenation unit 52 at dehydrogenation conditions to produce a methyl enhanced aromatic stream 54. The methyl enhanced aromatic stream 54 includes methyl enhanced aromatic compounds, such as xylenes and other methylated aromatic compounds than before the isomerization and dehydrogenation steps. “Methyl enhanced aromatic compounds,” as used herein, means aromatic compounds having more methyl groups connected to the aromatic ring. The methyl enhanced aromatic stream 54 is then passed to an aromatics complex 60 to isolate a desired xylene isomer. As such, the dehydrogenation unit 52 may be fluidly coupled to the isomerization unit 46 and to the aromatics complex 60. In alternate embodiments, the isomerization unit 46 is configured to produce the isomerized lignin stream 48, and the dehydrogenation unit 52 is configured to accept the isomerized lignin stream 48, so the isomerized lignin stream 48 may be transported from the isomerization unit 48 to the dehydrogenation unit 52 by rail car, barge, truck, or otherwise. The dehydrogenation unit 52 may include one or more reactors in series or parallel, so long as dehydrogenation conditions are maintained to dehydrogenate the isomerized lignin stream 48. The isomerization unit 46 produces some naphthenic compounds, and these compounds are dehydrogenated to form aromatics in the dehydrogenation unit 52. Dehydrogenation conditions in the dehydrogenation unit 52 include a pressure of from about 1 atmosphere to about 70 atmospheres in some embodiments, and a pressure of from about 3 atmospheres to about 30 atmospheres in other embodiments. The temperature in the dehydrogenation unit 52 may range from about 370° C. to about 570° C., where the temperature is typically increased over time to compensate for the inevitable deactivation of the dehydrogenation catalyst 56 that gradually occurs. Sufficient hydrogen is supplied to provide an amount of from about 1 to about 20 moles of hydrogen per mole of hydrocarbon in the isomerized lignin stream 48 used as the feed. In some embodiments, about 2 to about 10 moles of hydrogen are used in the dehydrogenation unit 52 per mole of hydrocarbon in the isomerized lignin stream 48. The liquid hourly space velocity used in dehydrogenation may be from about 0.2 to about 20 hr⁻¹.

Any suitable dehydrogenation catalyst 56 may be utilized in the dehydrogenation unit 52. In an exemplary embodiment, the dehydrogenation catalyst 56 includes a solid refractory oxide support with one or more platinum group metal components and optionally a modifier metal component such as tin or rhenium. This platinum-group component may exist within the final catalytic composite as a compound such as an oxide, sulfide, halide, oxyhalide, etc., in chemical combination with one or more of the other ingredients of the composite or as an elemental metal. However, in an exemplary embodiment, about 90 mass percent or more of the platinum-group component is present in the elemental state and is uniformly dispersed throughout the support material. This component may be present in the final dehydrogenation catalyst 56 in any amount which is catalytically effective. In many embodiments, relatively small amounts are effective, such as from about 0.01 mass percent to about 2 mass percent of the total mass of the dehydrogenation catalyst 56. The support can be any of a number of well-known supports in the art, including but not limited to aluminas, silica/alumina, silica, titania, zirconia, and zeolites. Included among the aluminas are aluminas which contain modifiers such as tin, zirconium, titanium and phosphate. The support can be formed in any desired shape such as spheres, pills, cakes, extrudates, powders, granules, etc. and it may be utilized in any particular size.

An IUPAC group 14 metal component is an optional ingredient of the dehydrogenation catalyst 56. This component may be present as an elemental metal, as a chemical compound such as the oxide, sulfide, halide, oxychloride, etc., or as a physical or chemical combination with the porous carrier material and/or other components of the dehydrogenation catalyst 56 composite. In an exemplary embodiment, about 70 mass percent or more of the IUPAC Group 14 metal exists in the finished catalyst in an oxidation state above that of the elemental metal. Rhenium is also an optional metal promoter of the dehydrogenation catalyst 56. In addition to the catalytic components described above, other components may be added to the dehydrogenation catalyst 56. For example, a modifier metal selected from the non-exclusive list of lead, indium, gallium, iridium, lanthanum, cerium, phosphorous, cobalt, nickel, iron and mixtures thereof may be added to the dehydrogenation catalyst 56. Yet another optional component of the dehydrogenation catalyst 56 is an alkali or alkaline-earth metal component. In an exemplary embodiment, this optional ingredient is one or more of cesium, rubidium, potassium, sodium, lithium, calcium, strontium, barium, and magnesium.

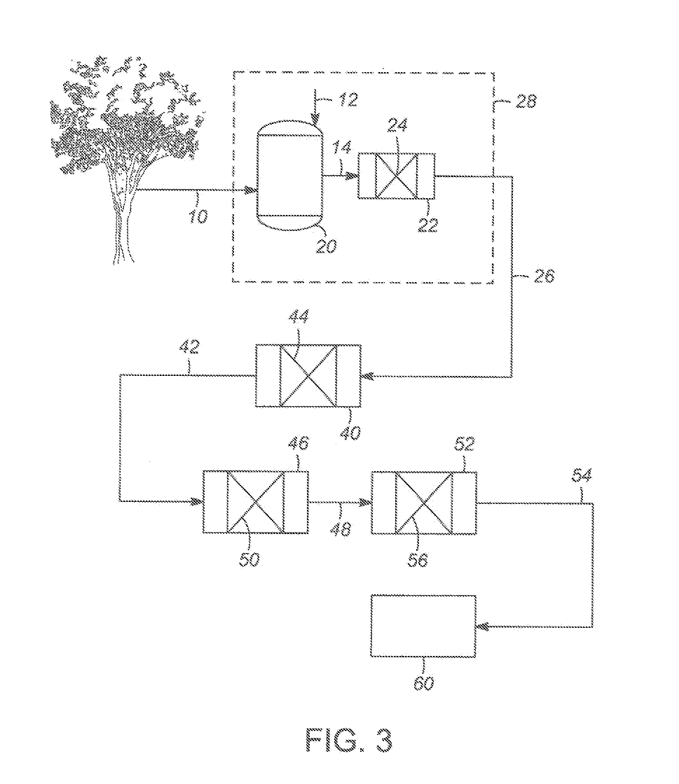

An alternate embodiment of the lignin depolymerization and isomerization process is illustrated in FIG. 3, where the lignin aromatic stream 26 is produced in the same manner as described above. In this alternate embodiment, the lignin aromatics stream 26 is treated in a hydrotreater 40, and the remaining oxygenates that may be present in the lignin aromatics stream 26 are removed to produce a hydrotreated lignin aromatic stream 42. As discussed above, the depolymerization of lignin can proceed to varying degrees of completeness, so some oxygenates may remain on the compounds in the lignin aromatic stream 26 in some embodiments. The oxygenates are not desired in the aromatics complex 60, so they are removed with the hydrotreater 40, where the hydrotreated lignin aromatics stream 42 may include elemental oxygen at a concentration of about 10 parts per million or less by weight. Hydrotreating is a well-known process, and involves contacting the lignin aromatics stream 26 and hydrogen gas with a hydrotreating catalyst 44 at hydrotreating conditions. In an exemplary embodiment, hydrotreating conditions in the hydrotreater 40 are a temperature of from about 290° C. to about 400° C., and a pressure of from about 20 to about 140 atmospheres. The reaction conditions are generally more severe as the hydrotreating catalyst 44 ages and becomes less active, and for hydrocarbon streams with higher boiling points, where more severe reaction conditions include higher temperatures and/or pressures. The oxygen reacts with the hydrogen gas to produce water, and sulfur compounds or nitrogen compounds that may be present in the lignin aromatics stream 26 may also be reacted to form hydrogen sulfide and ammonia, respectively. The gases (including water, hydrogen sulfide, and ammonia, if present) are then separated from the hydrotreated lignin aromatics stream 42, and excess hydrogen gas may be recovered and re-used in the hydrotreater 40.

In an exemplary embodiment, the hydrotreating catalyst 44 includes an IUPAC Group VI and/or IUPAC Group VIII active metal component on a support, where the support may be a porous refractory oxide including, but not limited to, alumina, alumina-silica, silica, zeolites, titania, zirconia, boria, magnesia, and their combinations. Supports other than refractory oxides are also possible in various embodiments. In some embodiments, other metals are included in the hydrotreating catalyst 44 in addition to or in place of the IUPAC Group VI and/or IUPAC Group VIII metals, such as cobalt, nickel, or other metals. For example, metals that may be used in the hydrotreating catalyst 44 include molybdenum, ruthenium, cobalt, nickel, tungsten, and combinations thereof. Hydrotreating catalysts 44 can be prepared by combining the active metals with the support. The supports, which may contain metal components, are typically dried and calcined at temperatures ranging from about 370° C. to about to 600° C. to eliminate any solvent and to convert metals to the oxide form, but other catalyst preparation processes are also possible. The calcined metal oxide catalysts may be reacted with sulfur to produce a metal sulfide, such as by contact with a sulfur containing compound including but not limited to hydrogen sulfide, organo sulfur compounds or elemental sulfur.

The hydrotreated lignin aromatics stream 42 is then introduced to the isomerization unit 46 and the dehydrogenation unit 52, as described for the lignin aromatics stream 26 described above and as illustrated in FIG. 1. The methyl enhanced aromatic stream 54 is produced in the dehydrogenation unit 52 and introduced into the aromatics complex 60, where a desired xylene isomer is isolated. High concentrations of naphthenes are undesirable in many aromatics complexes 60, and the optional hydrotreater 40 described above may convert some aromatics into naphthenes. However, the isomerization unit 46 and the dehydrogenation unit 52 are downstream from the hydrotreater 40, so naphthenes produced in the hydrotreater 40 are subsequently converted into aromatic compounds in the dehydrogenation unit 52.

The dehydrogenation unit 52 may be fluidly coupled to the aromatics complex 60 in some embodiments, but the methyl enhanced aromatic stream 54 produced in the dehydrogenation unit 52 may also be transported to the aromatics complex 60 in alternate embodiments, such as by rail car, barge, or truck. As such, the dehydrogenation unit 52 is configured to produce the methyl enhanced aromatic stream 54, and the aromatics complex 60 is configured to accept the methyl enhanced aromatic stream 54, so any manner of transferring the methyl enhanced aromatic stream 54 is acceptable. The lignin depolymerization unit 28, the lignin fractionator 30 if present, the isomerization unit 46, the dehydrogenation unit 52, and the aromatics complex 60 may each be remotely located or co-located in various embodiments.

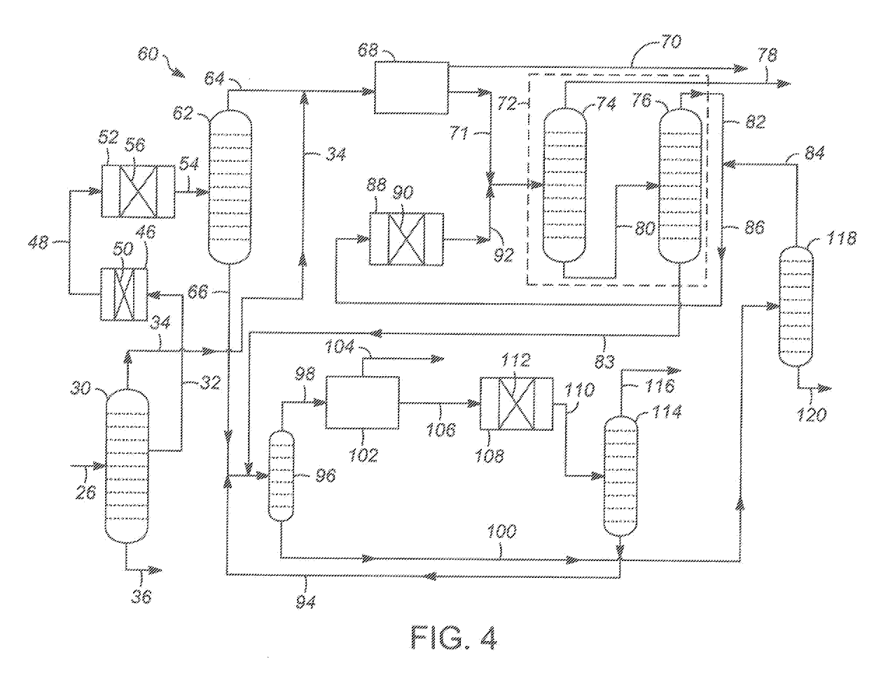

Reference is made to an exemplary embodiment of an aromatics complex 60 illustrated in FIG. 4, where other embodiments of an aromatics complex 60 are possible. The lignin fractionator 30 is illustrated, where the lignin center cut stream 32 flows to the isomerization unit 46, and the isomerized lignin stream 48 flows from the isomerization unit 46 to the dehydrogenation unit 52, as described above. The methyl enhanced aromatic stream 54 exits the dehydrogenation unit 52 and enters a reformate splitter column 62, where the reformate splitter column 62 is operated to produce a reformate splitter light ends stream 64 including toluene and lighter compounds, and a reformate splitter heavy ends stream 66 including xylenes and heavier compounds. The fractionators described below are operated at conditions appropriate to separate the components as described, as understood by those skilled in the art. The lignin light ends stream 34 is combined with the reformate splitter light ends stream 64 and directed to an optional aromatics separation unit 68. The aromatics separation unit 68 uses an extractive distillation process, a liquid-liquid extraction process, or a combination thereof to separate an aromatics free raffinate stream 70 from an A7-stream 71, where the A7-stream 71 includes aromatic compounds with 7 carbons or fewer. The aromatics free raffinate stream 70 is discharged from the aromatics complex 60, and may be used or further refined into paraffinic solvents, blended into gasoline, used as a feedstock for an ethylene plant, or otherwise used in a variety of manners.

The A7-stream 71 enters a fractionation zone 72 that may include one or more fractionators. In the embodiment illustrated, the fractionation zone 72 includes a benzene column 74 and a toluene column 76, but other configurations are also possible. The A7-stream 71 enters the benzene column 74, where it is split into a benzene stream 78 that primarily includes benzene, and an A7+ stream 80 that includes aromatic compounds with 7 or more carbon atoms. The A7+ stream 80 is fed to the toluene column 76 that is operated to produce a toluene stream 82 that primarily includes toluene, and an A8+ stream 83 that includes aromatic compounds with 8 carbons or more.

The toluene stream 82 is combined with a methyl-enriched A9-10 stream 84, described below, to produce a transalkylation feed stream 86. The A9-10 stream 84 primarily includes aromatic compounds with 9 or 10 carbon atoms that have been enriched with methyl groups, so the transalkylation feed stream 86 primarily includes toluene and methyl enriched A9-10 compounds, with a substantial absence of xylene (which is an A8 compound). The transalkylation feed stream 86 is introduced into a transalkylation unit 88 where it contacts a transalkylation catalyst 90 at transalkylation conditions.

The process continues by transalkylating the methyl enriched A9-10 compounds and the toluene in the transalkylation feed stream 86 to produce a transalkylation effluent 92. The transalkylation effluent 92 is optionally sent to a stripper column (not shown) within the transalkylation unit 88 to remove light ends, and then the transalkylation effluent 92 is recycled to the fractionation zone 72. The transalkylation unit light ends are benzene and other hydrocarbons with 6 carbons or less that may be used for various purposes, such as exported to a fuel gas system or recycled to the aromatics separation unit 68 to recover the benzene. The transalkylation unit 88 disproportionates toluene into benzene and mixed xylenes and transalkylates the methyl-enriched A9-10 compounds from the A9-10 stream with the toluene-containing stream into xylenes and benzene, as known in the art. The transalkylation unit 88 produced mixed xylenes from the toluene and A9-10 compounds, and the mixed xylenes exit the fractionation zone 72 in the A8+ stream 83 discharged from the bottom of the toluene column 76. The mixed xylenes are then processed to produce one or more desired xylene isomers, such as para-xylene, as described below.

The A8+ stream 83 is combined with the reformate splitter heavy ends stream 66 and a deheptanizer heavy ends stream 94, described below, and introduced into a xylenes column 96. The A8+ stream 83 and the reformate splitter heavy ends stream 66 include xylenes and heavier compounds, and the deheptanizer heavy ends stream 94 primarily includes xylenes, but may also include some toluene and some A9+ compounds. The xylenes column 96 produces a xylene stream 98 and xylene column heavy ends stream 100, where the xylene stream 98 primarily includes mixed xylenes, but may also include some toluene, and the xylene column heavy ends stream 100 includes A9+ compounds.

The xylenes stream 98 is introduced to the xylene recovery unit 102, where a desired xylene isomer stream 104 is separated from a xylene raffinate stream 106. Several techniques can be used for separating a desired xylene isomer stream 104, including selective adsorption and desorption, simulated moving bed adsorption, crystallization, ionic liquid separation, selective membranes, Etc. In an exemplary embodiment using selective adsorption, the desired xylene isomer stream 104 includes about 99% or greater para-xylene, along with a desorption solvent that can be separated from the para-xylene by distillation. The xylene raffinate stream 106 is almost entirely depleted of para-xylene, or the desired xylene isomer in embodiments where an isomer other than para-xylene is desired. The desorbent is recovered from the desired xylene isomer stream 104 and the xylene raffinate stream 106 by distillation (not illustrated), as understood by those skilled in the art. The desorbent may be heavier or lighter than the xylenes in various embodiments. In an exemplary embodiment, the xylene recovery unit 102 includes one or more adsorbent chambers with a selective adsorbent that preferentially adsorbs the desired xylene isomer over the other xylene isomers. In an exemplary embodiment, the selective adsorbent can be crystalline alumino-silicate, such as type X or type Y crystalline aluminosilicate zeolites. The selective adsorbent contains exchangeable cationic sites with one or more metal cations, where the metal cations can be one or more of lithium, potassium, beryllium, magnesium, calcium, strontium, barium, nickel, copper, silver, manganese, and cadmium. Adsorption conditions vary, but the temperature typically ranges from about 35° C. to about 200° C. and the pressure may be from about 1 atmosphere to about 35 atmospheres.

The xylene raffinate stream 106 is transferred to a xylene isomerization unit 108, where additional para-xylene (or other desired xylene isomer) is produced in a xylene isomer effluent stream 110 by re-establishing an equilibrium distribution of xylene isomers, as known to those skilled in the art. Other aromatics are also produced in lesser amounts. In an exemplary embodiment, the xylene isomerization unit 108 includes a xylene catalyst 112, and operates at suitable isomerization conditions. Suitable isomerization conditions include a temperature from about 100° C. to about 500° C., or from about 200° C. to about 400° C., and a pressure from about 4 atmospheres to about 50 atmospheres. The xylene isomerization unit 108 includes a sufficient volume of xylene catalyst 112 to provide a liquid hourly space velocity, with respect to the xylene raffinate stream 106, from about 0.5 to about 50 hr⁻¹, or from about 0.5 to about 20 hr⁻¹. Hydrogen may be present at up to about 15 moles of hydrogen per mole of xylene, but in some embodiments hydrogen is essentially absent from the xylene isomerization unit 108. The xylene isomerization unit 108 may include one, two, or more reactors, where suitable means are employed to ensure a suitable isomerization temperature at the entrance to each reactor. The xylenes are contacted with the xylene catalyst 112 in any suitable manner, including upward flow, downward flow, or radial flow. The xylene catalyst 112 and xylene isomerization unit 108 are essentially the same or similar to the isomerization unit 46 and the isomerization catalyst 50 used for the lignin aromatic stream 26 or lignin center cut stream 32 in some embodiments, but in other embodiments they are different.

In an exemplary embodiment, the xylene catalyst 112 includes a zeolitic aluminosilicate with a Si:Al₂ ratio greater than about 10/1, or greater than about 20/1 in some embodiments, and a pore diameter of about 5 to about 8 angstroms. Some examples of suitable zeolites include, but are not limited to, MFI, MEL, EUO, FER, MFS, MTT, MTW, TON, MOR, and FAU, and gallium may be present as a component of the crystal structure. In some embodiments, the Si:Ga₂ mole ratio is less than 500/1, or less than 100/1 in other embodiments. The proportion of zeolite in the xylene catalyst 112 is generally from about 1 to about 99 weight percent, or from about 25 to about 75 weight percent. In some embodiments, the xylene catalyst 112 includes about 0.01 to about 2 weight percent of one or more of ruthenium (Ru), rhodium (Rh), palladium (Pd), osmium (Os), Iridium (Ir), and platinum (Pt), but in other embodiments the xylene catalyst 112 is substantially absent of any metallic compound, where substantial absence is less than about 0.01 weight percent. The balance of the xylene catalyst 112 may be an inorganic oxide binder, such as alumina, and a wide variety of catalyst shapes can be used, including spherical or cylindrical.

The xylene isomer effluent stream 110 is transferred to a deheptanizer 114 that produces the deheptanizer heavy ends stream 94 and a deheptanizer light ends stream 116. The deheptanizer light ends stream 116 includes A6 compounds and other hydrocarbons with similar or lower boiling points. The deheptanizer light ends stream 116 may be split into a gas and a liquid product stream (not illustrated), where the gas may be exported to a fuel gas system and the liquid may be recycled back to the aromatics separation unit to recover benzene.

The xylene column heavy ends stream 100, described above, is transferred to a heavy aromatics column 118 that produces the A9-10 stream 84 that is combined with the toluene stream 82 from the toluene column 76. The heavy aromatics column 118 also produces an A11+ stream 120 that includes aromatics compounds with 11 carbon atoms or more. The A11+ stream 120 may be used or otherwise processed, such as for a feedstock for a fluid catalytic cracking unit.

The apparatus and method described above allows for the production of a desired xylene isomer from a renewable resource. The renewable resource, lignin, is also used to produce other valuable products such as benzene and various streams separated from the aromatics complex 60. Petroleum based feedstocks, such as a petroleum reformate, may be co-processed in the aromatics complex 60 with the methyl enhanced aromatic stream 54 in some embodiments (not illustrated). The petroleum based feedstock can be introduced to the reformate splitter column 62, the dehydrogenation unit 52, or at other locations. In other embodiments, the methyl enhanced aromatic stream 54 may be processed without any petroleum based feedstocks.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

1. A method of producing a desired xylene isomer from lignin comprising:
depolymerizing a lignin supply to produce a lignin aromatic stream;
isomerizing the lignin aromatic stream to produce an isomerized lignin stream; and
extracting the desired xylene isomer from the isomerized lignin stream.

2. The method of claim 1 further comprising:
fractionating the lignin aromatic stream to produce a lignin center cut stream, a lignin light ends stream, and a lignin heavy ends stream; and
wherein isomerizing the lignin aromatic stream comprises isomerizing the lignin center cut stream.

3. The method of claim 1 wherein extracting the desired xylene isomer comprises:
producing a desired xylene isomer stream and a xylene raffinate stream, wherein the desired xylene isomer stream comprises the desired xylene isomer; and
isomerizing the xylene raffinate stream in a xylene isomerization unit to produce the desired xylene isomer.

4. The method of claim 1 wherein depolymerizing the lignin supply comprises:
contacting the lignin supply with a lignin catalyst at depolymerization conditions, wherein the lignin catalyst comprises a metal.

5. The method of claim 1 further comprising:
dehydrogenating the isomerized lignin stream to produce a methyl enhanced aromatic stream; and wherein
extracting the desired xylene isomer from the isomerized lignin stream comprises extracting the desired xylene isomer from the methyl enhanced aromatic stream.

6. The method of claim 5 wherein isomerizing the lignin aromatic stream and dehydrogenating the isomerized lignin stream produces a methyl enhanced aromatic compound from propyl benzene.

7. The method of claim 6 further comprising:
producing xylene by transalkylating the methyl enhanced aromatic compound with toluene.

8. The method of claim 1 wherein depolymerizing the lignin supply comprises producing the lignin aromatic stream comprising propyl benzene in an amount greater than about 10 mole percent or more based on a total amount of aromatic compounds with 9 carbons in the lignin aromatic stream.

9. The method of claim 1 wherein depolymerizing the lignin supply comprises producing the lignin aromatic stream comprising propyl benzene in an amount greater than about 40 mole percent or more based on a total amount of aromatic compounds with 9 carbons in the lignin aromatic stream.

10. The method of claim 1 further comprising:
removing non-aromatic hydrocarbons from the isomerized lignin stream.

11. A method of producing xylene from lignin comprising:
depolymerizing lignin to produce a lignin aromatic stream;
hydrotreating the lignin aromatic stream to remove oxygen and to create a hydrotreated lignin aromatic stream;
contacting the hydrotreated lignin aromatic stream with an isomerization catalyst to produce an isomerized lignin stream; and
extracting the xylene from the isomerized lignin stream.

12. The method of claim 11 further comprising:
fractionating the hydrotreated lignin aromatic stream to produce a lignin center cut stream, a lignin light ends stream, and a lignin heavy ends stream; and
wherein contacting the hydrotreated lignin aromatic stream with the isomerization catalyst comprises contacting the lignin center cut stream with the isomerization catalyst.

13. The method of claim 11 wherein extracting the xylene comprises:
isolating a desired xylene isomer in a xylene recovery unit and producing a xylene raffinate stream; and
isomerizing the xylene raffinate stream in a xylene isomerization unit to produce the desired xylene isomer.

14. The method of claim 11 wherein depolymerizing the lignin comprises:
depolymerizing a lignin supply in the presence of a lignin catalyst, wherein the lignin catalyst comprises nickel, palladium, platinum, ruthenium, rhodium, molybdenum, cobalt, iron, tungsten, or a combination thereof.

15. The method of claim 11 further comprising:
contacting the isomerized lignin stream with a dehydrogenation catalyst to produce a methyl enhanced aromatic stream; and wherein
extracting the xylene from the isomerized lignin stream comprises extracting the xylene from the methyl enhanced aromatic stream.

16. The method of claim 15 wherein contacting the hydrotreated lignin aromatic stream with the isomerization catalyst and contacting the isomerized lignin stream with the dehydrogenation catalyst creates methyl enhanced aromatic compounds from propyl benzene.

17. The method of claim 16 further comprising:
producing xylene by transalkylating the methyl enhanced aromatic compounds with toluene.

18. The method of claim 11 wherein depolymerizing the lignin comprises producing the lignin aromatic stream comprising propyl benzene in an amount of greater than about 10 mole percent or more based on a total amount of aromatic compounds with 9 carbons in the lignin aromatic stream.

19. The method of claim 11 wherein depolymerizing the lignin comprises producing the lignin aromatic stream comprising propyl benzene in an amount of greater than about 40 mole percent or more based on a total amount of aromatic compounds with 9 carbons in the lignin aromatic stream.

20. An apparatus for producing a desired xylene isomer from lignin comprising:
a lignin depolymerization unit configured to produce a lignin aromatic stream;
an isomerization unit configured to accept the lignin aromatic stream, wherein the isomerization unit is further configured to produce an isomerized lignin stream;
a dehydrogenation unit configured to accept the isomerized lignin stream, wherein the dehydrogenation unit is configured to produce a methyl enhanced aromatic stream; and
an aromatics complex configured to accept the methyl enhanced aromatic stream, wherein the aromatics complex includes a xylene recovery unit, a transalkylation unit, and a xylene isomerization unit, and wherein the xylene recovery unit is configured to extract the desired xylene isomer.
